(19) United States
(12) Patent Application Publication
(10) Pub. No.: US 2022/0213407 A1
(43) Pub. Date: Jul. 7, 2022

(54) METHOD FOR LOWERING THE OXIDISING POWER OF A LIQUID OR SEMI-LIQUID ORGANIC COMPOSITION

(71) Applicant: LABIOCRAC, BRAINE (FR)

(72) Inventor: François ROLIN-MAAROUF, VILLENEUVE SAINT GERMAIN (FR)

(73) Assignee: LABIOCRAC, BRAINE (FR)

(21) Appl. No.: 17/602,444

(22) PCT Filed: Apr. 9, 2020
(86) PCT No.: PCT/EP2020/060289

§ 371 Date: Oct. 8, 2021

(30) Foreign Application Priority Data
Apr. 10, 2019 (FR) FR1903842

(51) Int. Cl. C11B 5/00; C09K 15/06; A61K 8/73; A61Q 19/00

(52) U.S. Cl. CPC C11B 5/0085; C09K 15/06; C11B 5/0021; A61K 2800/522; A61K 8/73; A61Q 19/00; A61K 2800/10; A61K 8/733

(57) ABSTRACT

A method for lowering the oxidising power of a liquid or semi-liquid organic composition, as well as of the so-called free organic composition that can be obtained by this method. Also, a free organic composition that can be obtained by the method of the invention, as well as a food, cosmetic or chemical composition or a pharmaceutical composition containing the composition treated by the method.

### TECHNICAL FIELD

The invention relates to a method for lowering the oxidizing power of a liquid or semi-liquid organic composition, as well as to the so-called free organic composition likely to be obtained by this method.

### PRIOR ART

Most of natural organic molecules are oxidizable in the presence of oxygen. Antioxidants are widely used as additives in the food industry to delay or even prevent oxidation of some molecules, which are responsible for food spoilage.

They are also used in many other fields such as plant refining, chemistry, cosmetics and pharmaceuticals. Antioxidants make it possible to control oxidation of organic molecules contained in the composition by limiting or even avoiding this oxidation. Examples of organic molecules that can be mentioned are polyphenols, proteins, lipids, vitamins, carotenoids, hormones, Adenosine Triphosphate (ATP), etc.

Indeed, from the moment when, in a food manufacturing method, the integrity of a seed, fruit or vegetable starts to be broken, for example by cutting, bleaching or grinding it, in order to suspend it in an aqueous solution, the enzymatic and/or chemical route oxidation and deterioration processes occur, leading to spoilage of products and co-products. In the food industry, oxidation generally leads to a degradation of organic molecules, appearing for example with the development of undesirable colors (typically brown or black), the change of taste and/or the transformation of active compounds into inactive compounds.

The same occurs in biomass extraction or refining methods, when an acidic or basic aqueous solution is added, with or without an enzyme, to solubilize molecules of interest contained in the biomass in order to extract them before purification.

The same phenomenon also occurs in aqueous, hydroalcoholic or oily compositions.

To overcome this oxidation problem in industry, an antioxidant or antioxidants such as SO₂, organic acids, vitamins such as vitamin C or vitamin E, etc. are usually added. The problem is that the antioxidants remain in the composition, which can cause health problems, for example because they generate intolerance. Further, antioxidants are controversial products in terms of their use in common commercial products.

Among the various mechanisms for combating oxidation of a composition that are commonly used today, the following ones can be mentioned:
Oxygen fixation or blocking. This is the mechanism of action of sulfites, glutathione or ascorbic acid. Sulfites are the subject of much controversy. Vitamin C (or ascorbic acid) eventually oxidizes, mainly in the presence of pro-oxidizing cations, and itself becomes sensitive to oxidation. The same applies to glutathione.
Radical chain blocking. This is the mode of action of vitamin E (or tocopherol) and its derivatives. Unfortunately, this action is also limited in time because this unstable molecule eventually oxidizes.

Acids, in particular citric acid or benzoic acid, slow down oxidation because they act on the polyphenol oxidase, which is inactive at an acidic pH and catalyzes oxidation reactions. However, this action is limited in time because acids solubilize the pro-oxidizing cations present in the composition and therefore do not completely stop oxidation.
Antioxidant polyphenols. The pro-oxidizing cations present in the composition eventually oxidize them, as a result of which they lose their antioxidant power. The same applies to carotenoids, ubiquinone, green tea, etc.

Application WO 2011/073941 A1 describes the use of a particular polymer capable of developing antioxidant properties in a composition under specific polymerization conditions. This polymer improves the antioxidant and anti-radical power of the composition. However, while it slows down conditions that cause oxidation, it does not completely block them. Ultimately, the pro-oxidizing cations present in the composition will oxidize the polymer.

Application WO 2015/067871 describes the use of pectic acid and/or its salts in soluble form to create a complex with a macromolecule having positively charged groups, said complex being made insoluble in aqueous medium and at an acidic pH. This application does not relate to oxidation phenomena in an organic composition. Moreover, pectic acid is implemented in the medium in soluble form.

Therefore, the solutions of the state of the art do not manage to satisfactorily solve the problems related to the use of antioxidants.

There is still a need for a new operating process of combating oxidation that does not have the drawbacks mentioned above.

### DISCLOSURE OF THE INVENTION

The purpose of the invention is to overcome the problems of the state of the art in the case of a liquid or semi-liquid organic composition. Said composition comprises at least pro-oxidizing cations and oxidation-sensitive molecules.

The innovative solution is described hereinafter.

A first object of the invention is a method for lowering the oxidizing power of a liquid or semi-liquid organic composition, said composition being aqueous, hydroalcoholic or oily, said method comprising the following successive steps of:

1) contacting at least one negatively charged organic polymer with the organic composition, said polymer being insoluble in said composition, said insoluble negatively charged organic polymer being selected from pectic acid and alginic acid, so as to obtain a preparation;

2) separating said polymer to which the pro-oxidizing cations present in the composition are bound, from the free organic composition, allowing the free organic composition to be recovered.

Advantageously, during said contacting, the pro-oxidizing cations, selected from the divalent and/or trivalent cations present in the composition which promote oxidation of the organic molecules present in said composition, bind to said insoluble negatively charged organic polymer.

During the separation step, the polymer to which the pro-oxidizing cations present in the composition have been bound is separated from the free organic composition.

The method according to the invention is based especially on a novel use of an insoluble negatively charged organic polymer to which the pro-oxidizing cations present in the composition bind, which makes them insoluble and thus unavailable to intervene in oxidizing the organic molecules present in said composition.

A second object of the invention is a free organic composition likely to be obtained according to the method of the invention.

A third object of the invention is a food, cosmetic or chemical composition comprising at least one free organic composition according to the invention.

A fourth object of the invention is a pharmaceutical composition comprising at least one free organic composition according to the invention, especially for its use as a drug.

### Definitions

By “organic», it is meant, according to the invention, containing carbon naturally or after synthesis.

By “Negatively charged organic polymer», it is meant, according to the invention, a macromolecular organic acid with a molecular weight of more than 5000 Dalton selected from pectic acid and/or alginic acid. It is a polymer which adsorbs cations.

By “pectic acid», it is meant, according to the invention, a pectic acid preferably in its acid form, generally with a degree of methylation preferably lower than 5%, in particular strictly lower than 5%. By “degree of methylation», it is meant the percentage of carboxyl groups of pectic acid which are in the form of —C(O)OCH₃ groups.

By “within the range of X to Y», it is meant that, according to the invention, it can take on any value from X to Y, including X and Y.

By “composition», it is meant, according to the invention, a product comprised of at least one chemical species, or a component.

By “plant origin”, is meant, according to the invention, any product derived from plants (cultured or wild) such as, for example, fruit, vegetables, seeds, aromatic plants and/or lignocellulosic plants. This product is generally in solid or liquid form. It can be, for example, either used in its raw form or suspended in an aqueous, hydroalcoholic or oily composition after grinding. This product may be intended for animal or human consumption, or for the cosmetic, chemical and pharmaceutical fields.

By “liquid composition», it is meant an essentially liquid composition including no solid element visible to the naked eye, that is with a size greater than about 0.2 mm when the element is located 20-25 cm from the observer.

By “Semi-liquid composition», it is meant a composition consisting of the mixture of a liquid component and a solid component, the solid component being generally in the form of particles visible to the naked eye.

By “Oxidizing power», it is meant, according to the invention, the capacity of the organic molecules contained in a composition to oxidize.

Oxidizing power is generally quantified by measuring conductivity. Conductivity is measured in a usual manner at ambient temperature, which is generally 20° C. but can be between 15° C. and 27° C., especially from 18° C. to 25° C., using a conductivity meter such as a bench-top conductivity meter. The measurement kinetics depend on the type of composition (biomass or other product) implemented in the method. The measurement kinetics can thus range from a few hours to a few days or even a few weeks depending on the composition implemented. For most compositions, it is sufficient to follow the kinetics over a week, or even a single day, as it reaches a plateau and does not change much after these times. Oxidation is considered to have stopped when the conductivity no longer increases when the conductivity kinetics has reached a plateau as discussed above. A color turning to brown/black of the organic composition is also an indicator of oxidation.

By “lowering the oxidizing power”, it is meant, according to the invention, that, over the time interval under consideration, the oxidizing power of the organic molecules contained in the free organic composition decreases, relative to the oxidizing power of the original liquid or semi-liquid organic composition. Preferably, the oxidizing power decreases by at least 50%, preferably by at least 80%, even more preferably by at least 90%.

By “pro-oxidizing cations», it is meant, according to the invention, the cations, in particular divalent and/or trivalent cations, present in a liquid or semi-liquid composition which promote oxidation of organic molecules present in said composition. By “promote oxidation», it is meant that said divalent and/or trivalent cations participate in the oxidation reactions of organic molecules in the presence of oxygen.

By “oily composition», it is meant, according to the invention, a preferably liquid composition comprising at least 50% (by volume) of oil(s). Such a composition may be in the form of a solution, that is a liquid containing a dissolved body, or an emulsion, that is a liquid preparation containing a suspended oily or resinous substance.

By “contacting», it is meant, according to the invention, incorporating the negatively charged organic polymer into the organic composition, generally by adding the polymer into the organic composition or by passing the composition through a polymer bed placed in a column-type container.

By “insoluble negatively charged organic polymer», it is meant, according to the invention, any negatively charged organic polymer which cannot dissolve in its medium, that is, for example, an aqueous or hydroalcoholic solution or an oily emulsion, regardless of the amount added at ambient temperature, that is, between 15° C. and 27° C., and especially between 18° C. and 25° C.

By “free organic composition», it is meant, according to the invention, a composition resulting from the method of the invention. According to the invention, this composition is such that most, that is at least 50%, in particular at least 80%, especially at least 90%, of the pro-oxidizing cations present in said initial composition have been removed by bonding with the negatively charged organic polymer and separated from said free organic composition.

Advantageously, the method according to the invention makes it possible to:
substantially block oxidation of oxidation-sensitive organic molecules present in the composition by virtue of the addition of the insoluble negatively charged organic polymer into said composition;
reduce the level of microbial contamination.

Indeed, the method of the invention makes it possible to insolubilize pro-oxidizing divalent and/or trivalent cations present in the organic composition by virtue of the insoluble negatively charged organic polymer which will bind and thus insolubilize these cations. In this way, these cations will no longer be pro-oxidizing. At the same time, microbial development is also limited because the divalent and/or trivalent cations necessary for their development are no longer available in the composition.

The pro-oxidizing cations according to the invention are generally selected from the group of divalent or trivalent cations of heavy metals such as, for example, iron or copper with several positive charges, which are likely to change valence. As pro-oxidizing cations for the purposes of the invention, mention may be made, for example, of metals such as iron, copper, calcium, magnesium, zinc, nickel, manganese and aluminum, which may be present, especially, in biomasses, or other metal cations such as barium, bismuth, mercury, lead, cadmium, cobalt, chromium or silicon.

The insoluble negatively charged organic polymer is selected from the group consisting of pectic acid (or polygalacturonic acid) and alginic acids, preferably pectic acid.

Pectic acid, which is a non-water soluble polymeric organic acid of galacturonic acid, is generally a product of pectin degradation by pectinase. It is a plant polymer rich in negatively charged free carboxylic functions.

As with pectins, pectic acids are characterized especially by a methylation level or degree of methylation (DM) which corresponds to the ratio of the carboxylic acid functions of the esterified (methylated) galacturonic acids to the total carboxylic acid functions of the galacturonic acids.

According to the invention, the preferred insoluble negatively charged organic polymer is pectic acid with a methylation level lower than 5%.

Pectic acid is generally found in powder form. It can therefore be used both bound to a column and stirred in a tank (“batch”).

Pectic acid can be easily removed in step 2) by solid-liquid separation. Thus, it is first introduced directly into the liquid or semi-liquid organic composition to be treated, and then, after pro-oxidizing cations of said composition have been bound, it is removed therefrom at the end of the method.

The liquid or semi-liquid composition is preferably selected from aqueous solutions, hydroalcoholic solutions, oily emulsions and semi-liquid compositions containing at least one biomass.

By “biomass», it is meant a mass of living matter remaining in equilibrium on a given surface of the earth globe.

Biomass is preferably of plant origin. It may have undergone one or more extractions of products of interest before being treated by using the method of the present invention.

The preferred liquid or semi-liquid organic compositions according to the invention are those containing organic molecules that are naturally oxidizable in the presence of oxygen dissolved in the liquid or semi-liquid composition. There can be mentioned for example: sunflower press cake, rapeseed press cake, aromatic plant solutions, fruit and vegetable dregs and skins, fruit or vegetable juices, wine, bleaching waters, cutting waters, vegetable oils, liquid cosmetic or pharmaceutical preparations. In accordance with the invention, prior to being contacted with an insoluble negatively charged organic polymer such as pectic acid or alginic acid, the biomass in solid form as mentioned above may undergo a treatment such as grinding, crushing, triturating or rasping, prior to or concomitantly with its dissolution, so that a liquid or semi-liquid organic composition is obtained, said composition being aqueous, hydro-alcoholic or oily.

Steps 1) and 2) are preferably carried out at a temperature in the range of 15 to 70° C., preferably in the range of 20 to 25° C. The pH of the composition prior to treatment with said organic polymer is generally between the values of 5 and 8.

The contacting step 1) is preferably carried out by at least one of the following actions: mixing, suspending, grinding, crushing, triturating, and rasping, as is known to the skilled person.

According to a preferred embodiment, said composition to be treated according to the present invention contains a biomass which is ground by using any means known to the skilled person, preferably a mechanical means.

Contacting the polymer with the liquid or semi-liquid organic composition according to step 1) may be done in a column or in a tank (“batch”, preferably with stirring).

The preparation obtained at the end of step 1) may possibly be mixed.

Preferably, the insoluble negatively charged organic polymer is present in the preparation from step 1) or step 2) in a content in the range of 1 to 30 g/L, in particular 1 to 15 g/L, especially 1 to 5 g/L, of preparation. This is true regardless of whether the preparation has been mixed or not.

Separating the insoluble negatively charged organic polymer of step 2) may be carried out by any conventional means, such as by centrifugation or filtration, as is known to the skilled person.

The method according to the invention is advantageously used in any industry in which the industrialist is confronted with the problem of unwanted oxidation of a liquid or semi-liquid organic composition, for example when the latter comprises oxygen dissolved in water.

There can be mentioned the food industry, plant refining, cosmetics, chemicals and pharmaceuticals. More specifically, the following compositions can be treated:
in the food industry: for example, green juice from sugar refinery; starch/gluten extraction juice, pectin extraction juice, rice steaming water, cutting and bleaching water and more generally process water in fruit and/or vegetable processing methods, fermented or non-fermented fruit or vegetable juices;
any extraction liquid (when processing is done after separation of the biomass from the liquid);
plant refining (or “biocracking”) products;
in the cosmetic field: water/oil or oil/water emulsions;
any pharmaceutical composition.

As previously indicated, products in solid form treated according to the present invention may undergo a treatment such as grinding, crushing, trituration or rasping before or concomitantly with their dissolution so that a liquid or semi-liquid organic composition is obtained, said composition being aqueous, hydroalcoholic or oily.

### BRIEF DESCRIPTION OF THE DRAWINGS

FIG. 1 represents the change in conductivity (C, in mS/cm) as a function of time (in Days, J) according to two exemplary embodiments (comparative and according to the invention) illustrating example 1.

FIG. 2 represents the change in conductivity (C, in mS/cm) as a function of time (in Days, J) according to two exemplary embodiments (comparative and according to the invention) illustrating example 2.

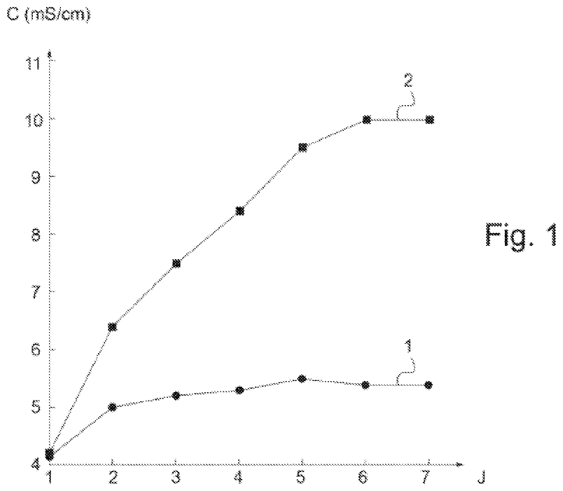

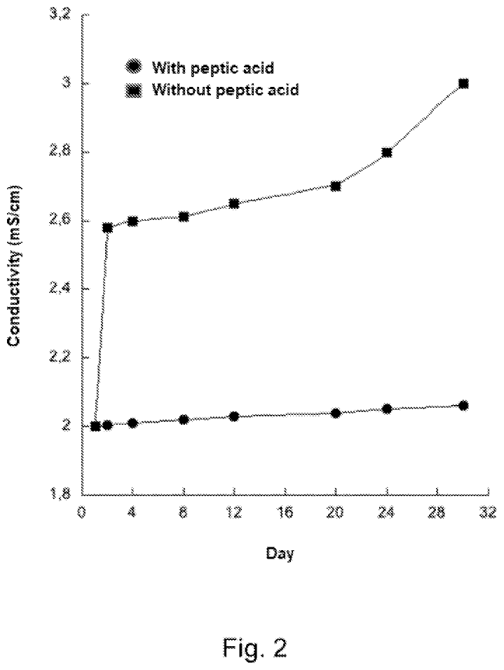

FIGS. 1 and 2 will be discussed hereinafter below in examples 1 and 2.

### EXAMPLES

### Example 1: Composition from Potato Treated with Pectic Acid

Potatoes contain chlorogenic acid, which is an oxidation-sensitive polyphenol. This tuber is therefore part of excellent examples for an oxidation test.

Test 1: Biomass without Addition of Insoluble Negatively Charged Polymer (for Comparison)

500 g of potato have been ground in 500 g of water in a knife mixer/grinder. The mixer/grinder has the advantage that it creates maximum oxidability conditions, as it allows air to be added to the ground material by foaming the proteins. After 10 minutes of mixing at speed 1, at a temperature of 20° C., the mixing has been stopped and a liquid-solid separation has been carried out by centrifugation. The extraction liquid thus obtained was rust/brown-colored, typical of an oxidized product.

The change in conductivity (C, in mS/cm) of the extraction liquid has been measured as a function of time (in Days, J) with a conductivity meter (Sanxin SX713) at 20° C., as represented in curve 2 of FIG. 1.

Test 2: Biomass Treated with the Addition of Insoluble Negatively Charged Polymer According to the Invention

500 g of potato was ground in 500 g of water to which 15 g of pectic acid was added in the same mixer as in Test 1. After 10 minutes of mixing at speed 1, at a temperature of 20° C., the mixing has been stopped and a liquid-solid separation has been carried out by centrifugation. The extraction liquid thus obtained was yellow in color. This yellow color, which remained stable for several days after the separation, demonstrated the absence of oxidation.

The change in conductivity (C, in mS/cm) of the extraction liquid has been measured as in Test 1 as a function of time (in Days, D), as represented in curve 1 of FIG. 1.

Unlike curve 2 of FIG. 1 corresponding to the comparative TEST 1, it is seen in curve 1 of FIG. 1 that the product treated by the method of the invention presents an asymptote at about 5.2 mS/cm, which demonstrated effectiveness of the method according to the invention since beyond 3 days and up to 7 days the oxidation of the product had not changed.

### Example 2: Sunflower Press Cake Composition Treated with Pectic Acid

Sunflower press cake de-oiled by pressing at 60° C. contains chlorogenic acid, which is another oxidation-sensitive polyphenol.

Test 1: Biomass without Addition of Insoluble Negatively Charged Polymer (for Comparison)

50 g of sunflower press cake de-oiled by pressing at 60° C. have been mixed using a magnetic stirrer in 1000 g of water at 25° C. After one hour of stirring, a solid-liquid separation has been carried out. The color of the supernatant started to turn black one hour after separation, which demonstrated oxidation.

Test 2: Biomass Treated with Addition of Insoluble Negatively Charged Polymer According to the Invention

50 g of sunflower press cake de-oiled by pressing at 60° C. have been mixed using a magnetic stirrer in 1000 g of water to which 12 g of pectic acid had been added at 25° C. After one hour of stirring, a solid-liquid separation has been carried out. The color of the supernatant remained pale yellow even several days after separation, which demonstrated that the polyphenol had not oxidized.

This demonstrated the effectiveness of the method according to the invention, in comparison with the result of the comparative test 1.

### Example 3: Apple Juice Composition Treated with Pectic Acid

The apple juice contains chlorogenic acid, which is an oxidation-sensitive polyphenol. Conductivity has been measured to determine the oxidizing power of the treated composition in comparison with that not treated by the addition of pectic acid.

Test 1: Biomass without Addition of Insoluble Negatively Charged Polymer (for Comparison)

1 L of juice has been extracted from apples at ambient temperature using a household juice extractor. The obtained apple juice has been filtered to remove large solid particles and then pasteurized by heating to 70° C. for 30 minutes.

Test 2: Biomass Treated with Insoluble Negatively Charged Polymer According to the Invention

1 L of juice has been extracted from apples at ambient temperature, using a household juice extractor. The apple juice obtained has been mixed with 10 g/L of insoluble pectic acid to obtain a pH of 3.5. The obtained composition was then filtered to remove large solid particles and then pasteurized by heating to 70° C. for 30 minutes.

For TESTS 1 and 2, the following measurements have been made:

Conductivity Measurement

The change in oxidation is monitored by measuring the conductivity (in mS/cm) of the extraction liquid as a function of time (in days), using a conductivity meter (Sanxin SX713) at 20° C.

Bacterial Contamination Measurement:

Bacterial concentration measurements (enterobacteria) have also been carried out at T0 and T+5 days.

In this test, the French method according to NF V08-054-04/2009 standard has been used to enumerate presumed enterobacteria by counting colonies obtained at 30° C. or 37° C., in products intended for human consumption or animal feed.

The results are reported in FIG. 2:

Conductivity:

For the sample not treated with pectic acid (“without pectic acid” curve) it is noted that the conductivity increases very rapidly and strongly during the first day starting from an initial value of 2 mS/cm up to 2.8 mS/cm, and then progressively increases to the value of 3 mS/cm for more than 30 additional days.

In contrast, for the sample treated with pectic acid, according to the method of the invention (“with pectic acid” curve), a quasi-stability of conductivity to the initial value (2 mS/cm) is noted during the whole period of the test (30 days).

These results show very clearly that in the case of the apple juice sample treated with pectic acid, according to the invention, oxidation was blocked.

Bacterial Contamination

A bacterial concentration (enterobacteria) at D+5 of 120,000 CFU/g is noticed for the juice not treated with pectic acid, in comparison with <100 CFU/d for the juice treated with pectic acid according to the invention.

This result clearly shows a highly reduced bacterial contamination when the apple juice has been treated with pectic acid.

### Example 4: Composition of Sunflower Press Cake Treated with Alginic Acid

A sunflower press cake de-oiled by pressing at 60° C. contains chlorogenic acid, which is an oxidation-sensitive polyphenol.

Test 1: Biomass without Addition of Insoluble Negatively Charged Polymer (for Comparison)

50 g of sunflower press cake have been mixed using a magnetic stirrer in 1000 g of water at 25° C. After one hour of stirring, a solid-liquid separation has been carried out. The color of the supernatant started to turn black one hour after separation, which demonstrated oxidation.

Test 2: Biomass Treated with Addition of Insoluble Negatively Charged Polymer According to the Invention

50 g of sunflower press cake have been mixed using a magnetic stirrer in 1000 g of water to which 25 g of alginic acid had been added at 25° C. After one hour of stirring, a solid-liquid separation has been carried out. The color of the supernatant remained pale yellow even several days after separation, which demonstrated that the polyphenol had not oxidized.

This demonstrated the effectiveness of the method according to the invention, in comparison with the result of the comparative test 1.

1-15. (canceled)

16. A method for lowering the oxidizing power of a liquid or semi-liquid organic composition, said composition being aqueous, hydroalcoholic or oily and comprising at least pro-oxidizing cations and oxidation-sensitive molecules, said method comprising the following successive steps of:
1) contacting at least one negatively charged organic polymer with the organic composition, said polymer being insoluble in said composition, said insoluble negatively charged organic polymer being selected from pectic acid and alginic acid, so as to obtain a preparation;
2) separating said polymer to which the pro-oxidizing cations present in the composition are bound, from the free organic composition, allowing the free organic composition to be recovered.

17. The method according to claim 16, wherein, during contacting in step 1), the pro-oxidizing cations, selected from the divalent and/or trivalent cations present in the composition which promote oxidation of the organic molecules present in said composition, bind to said insoluble negatively charged organic polymer.

18. The method according to claim 16, wherein the preparation obtained after step 1) is mixed.

19. The method according to claim 16, wherein pectic acid has a methylation level lower than 5%.

20. The method according to claim 16, wherein the liquid or semi-liquid organic composition contains at least one biomass, preferably of plant origin.

21. The method according to claim 16, wherein the biomass is ground before being added to the composition.

22. The method according to claim 16, wherein each of steps 1) and 2) is carried out at a temperature in the range of 15 to 70° C., preferably in the range of 20 to 25° C.

23. The method according to claim 16, wherein the contacting step 1) is carried out by at least one of the following actions: mixing, suspending, grinding, crushing, trituration or rasping.

24. The method according to claim 16, wherein in step 2), pectic acid or alginic acid is removed by solid-liquid separation.

25. The method according to claim 16, wherein the insoluble negatively charged organic polymer, which is pectic acid or alginic acid, is present in the preparation in a content in the range from 1 to 30 g/L, in particular 1 to 15 g/L, especially 1 to 5 g/L of preparation.

26. The method according to claim 16, wherein the separation step 2) is carried out by centrifugation or filtration.

27. The method according to claim 16, wherein the insoluble negatively charged organic polymer, which is pectic acid or alginic acid, is bound to a column.

28. The method according to claim 16, wherein the oxidizing power of the organic molecules contained in the free organic composition is reduced by at least 50%, preferably by at least 80%, even more preferably by at least 90%, relative to the liquid or semi-liquid organic composition used in step 1).

29. A free organic composition having a lowered oxidizing power likely to be obtained according to claim 16.

30. A food, cosmetic or chemical composition comprising at least one free organic composition according to claim 29.
